(19) United States
(12) Patent Application Publication
(10) Pub. No.: US 2024/0353402 A1
(43) Pub. Date: Oct. 24, 2024

(54) RAPID TICK TESTER

(71) Applicant: TBP Diagnostics, LLC, Willington, CT (US)

(72) Inventors: Lawrence Silbart, Willington, CT (US); Thanh Nguyen, Storrs, CT (US)

(21) Appl. No.: 18/643,170

(22) Filed: Apr. 23, 2024

Related U.S. Application Data
(60) Provisional application No. 63/461,358, filed on Apr. 24, 2023.

(51) Int. Cl. G01N 33/543; G01N 33/53; G01N 33/569

(52) U.S. Cl. CPC G01N 33/54388; G01N 33/5308; G01N 33/56905; G01N 33/56911; G01N 33/56983; G01N 2333/08; G01N 2333/11; G01N 2333/183; G01N 2333/195; G01N 2333/20; G01N 2469/10

(57) ABSTRACT

Systems and methods are described herein for a diluent-containing tube comprising a beveled grinding surface and operable to receive a plunger comprising a complementary beveled grinding surface. Solid matrices placed within the diluent can be ground between the grinding surfaces to release molecules into the diluent for detection. The plunger may further comprise a groove containing a test strip operable to be deployed into the diluent after grinding for detection of the released molecules. In certain embodiments, the test strip may be a lateral flow immunoassay rapid antigen detection (LFA) test strip. The solid matrix may be a tick and the molecules may be antigens or pathogens related to tick-borne illnesses.

### CROSS REFERENCE TO RELATED APPLICATIONS

This application claims priority to and benefit of U.S. Provisional Application Ser. No. 63/461,358, filed Apr. 24, 2023, the content of which is incorporated herein by reference in its entirety.

### TECHNICAL FIELD

This application relates generally to systems, methods and apparatuses, for rapid testing and identification of molecules in a solid matrix.

### BACKGROUND

The incidence of tick-borne diseases is increasing throughout the United States and in many other regions of the world due in part due to global warming. This is manifest in two ways: a wider geographic distribution over which ticks and their vertebrate hosts can thrive, paired with milder winters, which allows a higher proportion of ticks to survive. As housing subdivisions push farther into forested areas, the proximity to tick-infested areas and wildlife places more humans at risk. Moreover, a higher proportion of ticks are carrying pathogenic organisms, and many now carry two or more disease agents at a time, resulting in co-infection of humans and animals.

Once a blood-engorged tick is detected and removed from a human or companion animal, the ensuing days can be fraught with anxiety and uncertainty since the infection status of the tick is unknown. Many diagnostic laboratories have the capacity to test ticks using highly sensitive methods including PCR or immunohistochemical testing. This requires that the tick be properly stored, then mailed or transported to a tick-testing lab. This service is sometimes free, but in other cases can cost upward of $100. Regrettably, the turn-around time for these lab tests is 48- to 72-hours from submission, resulting in the loss of precious time during which the bacterium replicates in the skin, then migrates via blood and lymphatics to establish a disseminated infection culminating in Lyme Disease, Ehrlichiosis, Anaplasmosis and other tick-borne diseases. Thus, a critical treatment window is lost while test results are pending.

While some physicians prescribe prophylactic antibiotics after removal of an engorged tick, others consider this practice to be misguided since the tick may be negative for pathogens, may not have transmitted the pathogen in sufficient quantities to cause an infection, and many patients have adverse reactions to antibiotics including allergic responses and intestinal dysbiosis. It is also widely accepted that over-prescribing antibiotics leads to the vexing problem of microbial antibiotic resistance, a worldwide problem. Thus, most patients go untreated until symptoms arise (e.g., a bullseye rash), at which time they seek treatment. Regrettably, many individuals do not develop the typical bullseye rash, and weeks or months later develop intractable Lyme disease which is difficult to diagnosis, and expensive to treat.

Approximately 20% of the 500,000 Americans per year who contract Lyme Disease do not clear the infection, and develop severe, long-term, and sometimes life-threatening chronic disease. In many instances this can occur even after antibiotic treatment, leading to ‘post-treatment Lyme Disease syndrome.’ Thus, a rapid diagnostic platform for assessing the infection status of the tick, as described herein, is urgently needed.

### SUMMARY

The device described herein assesses the infection status of the tick, not the human or vertebrate host. It will be used for several purposes, primarily to assess potential exposure to a pathogen, but also for geospatial mapping to guide hikers and outdoorsman regarding where pathogen-bearing ticks have been found. Additionally, the device may be used for ‘citizen science’ projects to determine pathogen prevalence and to assess the proportion of ticks bearing one or more pathogens simultaneously.

Embodiments of the device described herein allow the user to safely grind the tick using a non-toxic diluent solution, then assess infection status based on an integrated lateral flow immunoassay (LFA). FIG. 1 shows this self-contained unit in its entirety.

One embodiment is a single-use, disposable integrated tick-testing device comprising a plunger with a beveled tip representing one of two grinding surfaces, a diluent-containing receiver tube with a beveled bottom which mates up with the beveled surface of the plunger and provides a grinding surface at the bottom and sides of the receiving tube.

One embodiment is a tick-testing device where the tick is placed into the receiver tube and mashed dry. After mashing diluent fluid is added, additional mashing is performed, and the diluent is either transferred to an LFA test strip using a supplied dropper, or an integrated LFA test strip is repositioned to contact the diluent at the bottom of the receiver tube.

One embodiment is a tick-testing device where the tick is placed in a non-toxic diluent and ground wet. After grinding, the diluent (containing released pathogens) may be transferred to an LFA test strip using a supplied dropper.

In one embodiment, the tick-testing device is effective to test for at least one tick-borne disease. In other embodiments, the tick-testing device is effective to test two or more tick-borne diseases simultaneously.

In one embodiment, after the tick has been ground, an integrated LFA test strip will be repositioned within an embedded slot in the plunger body, allowing a sample pad to contact the diluent. The sample is then wicked into the pad, at which time it passes over a conjugate pad, allowing antigenic moieties on the surface of the bacterium to interact with pathogen-specific antibodies bound to a detection particle. Once bound to the conjugate, the material travels with the diluent through a nitrocellulose test strip, passing over pathogen-specific antibodies immobilized in a line or dot at a specific location on the nitrocellulose test strip. The immobilized antibodies capture the conjugate-tagged pathogen when it is present. No test line is observed in the absence of the specific pathogen. As more and more conjugate-tagged bacteria collect where the immobilized specific antibody has been deposited, a visible line or dot becomes apparent. Fluid will continue to flow through the test strip into an absorbent pad, allowing the device to wick nearly the entire diluent volume over the conjugate pad and test strip as a ‘flow through’ device. Test lines containing immobilized pathogen-specific capture antibodies are labeled so that the user can assess which pathogen has been detected (e.g., Bb—Borrelia; EL—Ehrlichia; An—Anaplasma). A positive control ‘C’ line is included to ensure that the test was performed properly.

Other aspects of the invention will become apparent by consideration of the detailed description and accompanying drawings.

In certain aspects, a testing device is described comprising a plunger including a groove extending along a length of the plunger and a beveled tip, the beveled tip comprising a first grinding surface. The testing device can further comprise a diluent-containing receiving tube having a beveled bottom, wherein the beveled bottom provides a second grinding surface and wherein the receiving tube is operable to receive the plunger such that the first grinding surface of the beveled top contacts the second grinding surface of the beveled bottom. Testing devices of the invention can further comprise a test strip positioned in the groove of the plunger. The test strip may be slidably positioned in the groove of the plunger and operable, upon fully inserting the plunger into the tube, to be slid into and out of the diluent within the tube.

In various embodiments, the test strip may be a lateral flow immunoassay rapid antigen detection (LFA) test strip. The LFA test strip can comprise one or more high-affinity binding partners operable to bind and detect an antigen. The one or more high-affinity binding partners can comprise an antibody. The one or more high-affinity binding partners may comprise an aptamer. In some cases, the detection method may include PCR, FISH or other molecular approaches for detecting an antigen wherein the detecting moiety includes a nucleic acid probe. In certain embodiments, the LFA test strip may be operable to detect one or more diseases or disease-causing antigens selected from the group consisting of Borrelia sp., Ehrlichia sp, Anaplasma sp., Rocky Mountain spotted fever, Rickettsia sp., tularemia, Babesia sp., heartland virus, bourbon virus, Colorado tick fever, Powassan virus disease, relapsing fever, Mediterranean spotted fever, R. conorii, tick-borne encephalitis (TBE Virus), neoehrlichiosis, and Lyme-like illness.

In some embodiments, repeated full insertion of the plunger into the receiving tube can be operable to grind a tick placed into the receiving tube between the first and second grinding surfaces sufficiently to release the antigen from the tick into the diluent. Testing device of the invention may be operable to release and detect Cry9C, exogenous transgenes, small molecules, pesticides, microbial toxins, aflatoxins, universal plant stress proteins, heat-shock proteins, antioxidant enzymes, antimicrobial peptides, delta 8- or delta-9 tetrahydrocannabinol, opium, nicotine or quorum-sensing signals, from food items or plants placed in the receiving tube and ground between the first and second grinding surfaces therein.

Aspects of the invention may include methods of releasing molecules embedded in a solid matrix, the methods comprising: providing a testing device comprising: a plunger comprising: a groove extending along a length of the plunger, and a beveled tip, the beveled tip comprising a first grinding surface; and a diluent-containing receiving tube having a beveled bottom, wherein the beveled bottom provides a second grinding surface; placing the solid matrix into the diluent-containing receiving tube; inserting the plunger into the diluent-containing receiving tube; and grinding the solid matrix between the first grinding surface of the beveled tip and the beveled bottom, thereby releasing molecules embedded in the solid matrix into the diluent for detection. Further methods of using the testing devices described herein are contemplated and will be apparent to those of ordinary skill in the art.

### BRIEF DESCRIPTION OF THE DRAWINGS

The following drawings are included to illustrate certain embodiments of the invention:

FIG. 1 illustrates one embodiment of a disposable integrated tick-testing device.

FIG. 2 illustrates a portion of a disposable integrated tick-testing device.

FIG. 3 illustrates a separate portion of a disposable integrated tick-testing device.

### DETAILED DESCRIPTION

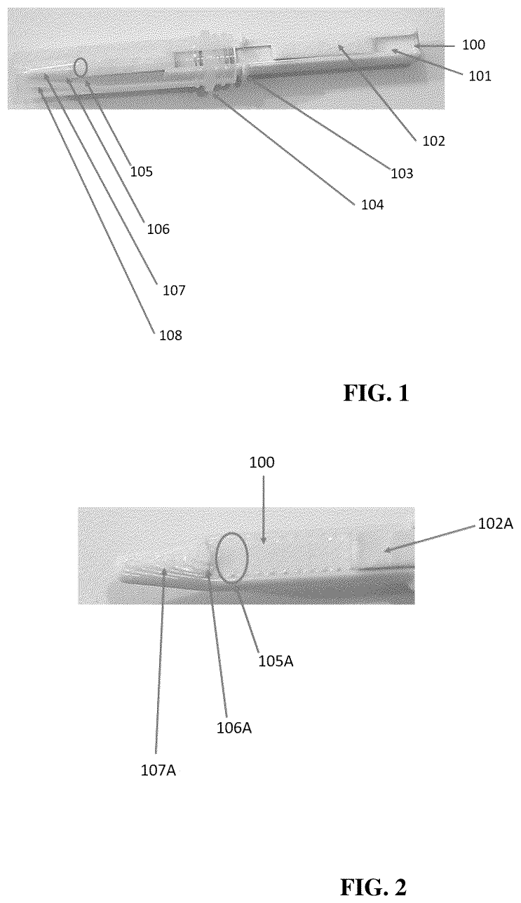

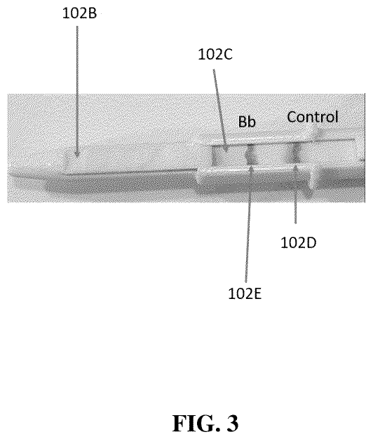

As shown in FIG. 1, in one embodiment, the single-use, disposable integrated tick-testing device comprises a plunger [100] with a beveled tip [107] representing one of two grinding surfaces, a diluent-containing receiver tube [104] with a beveled bottom [108] which mates up with the beveled surface of the plunger in such a way as to minimize any ‘dead space’. When the plunger is fully inserted into the receiver tube, the diluent is displaced into the fluid reservoir [105]. Together, these items comprise the tick grinding chamber. The final component is a lateral flow immunoassay (LFA) rapid antigen detection test strip [102] which is pre-loaded into a groove [101] in the plunger body.

In one embodiment, the tick-testing device is made from a material suitable to withstand packaging, shipping and use by a consumer. While the invention is not limited in this regard, and is contemplated to be made by one or many different types of material, it is envisioned that in one embodiment, the tick-testing device is made from a polymeric material, i.e., plastic. In one embodiment, the invention is made from plastic such as polypropylene, polyester, polyethylene, or a combination of any of the foregoing. In one embodiment, the tick-testing device is made from virgin plastic. In one embodiment, the tick-testing device is made from recycled plastic. In one embodiment, the tick-testing device includes virgin plastic and recycled plastic. Other materials, such as plant-based materials, may be used either alone or in combination with other materials.

The dimensions suitable for the tick-testing device vary and are not seen as a limiting feature of the present invention. In one embodiment, the tick-testing device has a height of about 3.5 inches when freestanding in an upright position. In another embodiment, the tick-testing device has a height of between about 3.0 inches to about 4.0 inches. Other lengths outside the ranges provided herein are contemplated. In one embodiment, the tick testing device has a width of about 0.50 inch, when measured across its face when the device is freestanding in an upright position. The circumference of the device is approximately 1.75 inches at its widest point. In one embodiment, the height, width, and circumference of the tick-testing device may be variable.

The tick-testing device is constructed in a user-friendly manner and is operated as follows. The native or engorged tick is dropped into the diluent-containing receiver tube and the plunger [100] is inserted. The user then grinds the tick by making successive up-and-down motions with the plunger, thereby ensuring that the bacteria within the tick are released into the diluent. During the final compression, the fluid overruns the beveled portion of the plunger, entering a fluid reservoir shown in FIG. 2 [105A]. The LFA strip [102A] is then gently lowered into the diluent by sliding it down the channel until it comes to a stop [106, 106A], at which time the fluid in the reservoir [105A] contacts the LFA sample pad [102B]. The diluent is immediately wicked into the LFA sample pad [102B] via capillary action. Any solid material will be entrapped in the sample pad, allowing the fluid to pass over the conjugate pad (not shown) and into the nitrocellulose test strip [102C], eventually flowing over the detection lines [102D, 102E], illuminating the positive control line, and then potentially illuminating one of the test lines based on the presence or absence of a specific pathogen. A splash guard [103], with or without a rubber O-ring (not shown) is integrated into the plunger body to prevent fluid escape during the grinding process. Five- to fifteen minutes after grinding, the results can be assessed, and the device is either discarded in household trash or mailed to a pre-determined testing lab for validation.

In another embodiment, the tick is placed into the receiver tube and mashed dry with a plunger. After mashing the plunger is partially withdrawn and 3 drops of diluent fluid is transferred using a supplied dropper. A final mixing is performed before lowering the test strip into position, at which time the fluid enters the LFA sample pad as described above.

In another embodiment, the test strip can be manufactured to detect specific pathogens in different geographic locations. For example, for the Northeast, Eastern Seaboard and upper Midwest (USA), the strip would be optimized for Borrelia sp., Ehrlichia sp, and Anaplasma sp., whereas in the Western Unites States, Rocky Mountain spotted fever could be added or substituted. Other tick-borne pathogens includes, but are not limited to the following pathogens/diseases:
Rickettsia species
Tularemia
Babesia
Heartland and Bourbon Virus
Colorado Tick Fever
Powassan Virus Disease

In another embodiment, additional tick-borne disease agents found in other regions of the world may be developed, including, but not limited to those listed above plus:
Relapsing fever (Borrelia species)
Mediterranean spotted fever (R. conorii)
Tick-borne encephalitis (TBE Virus)
Neoehrlichiosis
Lyme-like illness

In another embodiment, the device can be used to detect other proteins or pathogens entrapped or embedded in a solid matrix including food items or plants. This includes but is not limited to the following: genetically modified plants containing Cry9C or other exogenous transgenes, small molecules (e.g., pesticides or microbial toxins such as aflatoxins), select agents and pathogens, universal plant stress proteins (heat-shock proteins, antioxidant enzymes, antimicrobial peptides etc.), or quorum-sensing signals, all of which may be entrapped in a solid matrix. The plant material would be inserted into the grinding apparatus and the LFA test strip would be used in a conventional solid phase immunoassay format as described, or in a competitive immunoassay for small molecules (e.g., delta 8- or delta-9 tetrahydrocannabinol, opium, nicotine, plus those listed above etc.).

In various embodiments, the LFA test strip may use specific antibody capture or other molecular approaches to high-affinity binding partners including aptamer technology or cloned antibody fragments wherein the high-affinity binding partner(s) selectively binds and signals the presence of an antigen or molecule of interest. Lateral flow assays are known in the art and described generally, for example, in Omidfar, et al., 2023, Lateral Flow Assay: A Summary of Recent Progress for Improving Assay Performance, Biosensors, 13:837, the content of which is incorporated herein by reference in its entirety. Exemplary aptamer detection methods are described, for example, in U.S. Pat. No. 11,300,519, the content of which is incorporated herein by reference in its entirety. In some embodiments molecular approaches may be used for detecting the nucleic acid content of pathogens including PCR, fluorescent in situ hybridization, or other methods using nucleic acid probes including labeled nucleic acid probes.

As will be apparent to those skilled in the art, various modifications, adaptations, and variations of the foregoing specific disclosure can be made without departing from the scope of the invention claimed herein. The various features and elements of the invention described herein may be combined in a manner different than the specific examples described or claimed herein without departing from the scope of the invention. In other words, any element or feature may be combined with any other element or feature in different embodiments, unless there is an obvious or inherent incompatibility between the two, or it is specifically excluded.

References in the specification to “one embodiment,” “an embodiment,” etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such aspect, feature, structure, or characteristic with other embodiments, whether or not explicitly described.

The singular forms “a,” “an,” and “the” include plural reference unless the context clearly dictates otherwise. Thus, for example, a reference to “a plant” includes a plurality of such plants. It is further noted that the claims may be drafted to exclude any optional element. As such, this statement is intended to serve as antecedent basis for the use of exclusive terminology, such as “solely.” “only,” and the like, in connection with the recitation of claim elements or use of a “negative” limitation. The terms “preferably,” “preferred.” “prefer.” “optionally.” “may.” and similar terms are used to indicate that an item, condition or step being referred to is an optional (not required) feature of the invention.

The term “and/or” means any one of the items, any combination of the items, or all of the items with which this term is associated. The phrase “one or more” is readily understood by one of skill in the art, particularly when read in context of its usage.

Each numerical or measured value in this specification is modified by the term “about” The term “about” can refer to a variation of ±5%, ±10%, ±20%, or ±25% of the value specified. For example, “about 50” percent can in some embodiments carry a variation from 45 to SS percent. For integer ranges, the term “about” can include one or two integers greater than and/or less than a recited integer at each end of the range. Unless indicated otherwise herein, the term “about” is intended to include values and ranges proximate to the recited range that are equivalent in terms of the functionality of the composition, or the embodiment.

As will be understood by the skilled artisan, all numbers, including those expressing quantities of reagents or ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all language such as “up to”, “at least”, “greater than”, “less than”. “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only, they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

What is claimed is:

1. A testing device comprising:
a plunger including:
a groove extending along a length of the plunger, and
a beveled tip, the beveled tip comprising a first grinding surface; and
a diluent-containing receiving tube having a beveled bottom, wherein the beveled bottom provides a second grinding surface and wherein the receiving tube is operable to receive the plunger such that the first grinding surface of the beveled top contacts the second grinding surface of the beveled bottom.

2. The testing device of claim 1, further comprising a test strip positioned in the groove of the plunger.

3. The tick-testing device of claim 2, wherein the test strip is slidably positioned in the groove of the plunger and operable, upon fully inserting the plunger into the tube, to be slid into and out of the diluent within the tube.

4. The testing device of claim 2, wherein the test strip is a lateral flow immunoassay rapid antigen detection (LFA) test strip.

5. The testing device of claim 4, wherein the LFA test strip comprises one or more high-affinity binding partners operable to bind and detect an antigen or portion thereof.

6. The testing device of claim 5, wherein the one or more high-affinity binding partners comprise an antibody.

7. The testing device of claim 5, wherein the one or more high-affinity binding partners comprise an aptamer or a nucleic acid probe.

8. The testing device of claim 5, wherein the LFA test strip is operable to detect one or more diseases or disease-causing antigens selected from the group consisting of Borrelia sp., Ehrlichia sp, Anaplasma sp., Rocky Mountain spotted fever, Rickettsia sp., tularemia, Babesia sp., heartland virus, bourbon virus, Colorado tick fever, Powassan virus disease, relapsing fever, Mediterranean spotted fever, R. conorii, tick-borne encephalitis (TBE Virus), neoehrlichiosis, and Lyme-like illness.

9. The testing device of claim 5, wherein repeated full insertion of the plunger into the receiving tube is operable to grind a tick placed into the receiving tube between the first and second grinding surfaces sufficiently to release the antigen from the tick into the diluent.

10. The testing device of claim 5, operable to release and detect Cry9C, exogenous transgenes, small molecules, pesticides, microbial toxins, aflatoxins, universal plant stress proteins, heat-shock proteins, antioxidant enzymes, antimicrobial peptides, delta 8- or delta-9 tetrahydrocannabinol, opium, nicotine or quorum-sensing signals, from food items or plants placed in the receiving tube and ground between the first and second grinding surfaces therein.

11. A method of releasing molecules embedded in a solid matrix, the method comprising:
providing a testing device comprising:
a plunger comprising:
a groove extending along a length of the plunger, and
a beveled tip, the beveled tip comprising a first grinding surface; and
a diluent-containing receiving tube having a beveled bottom, wherein the beveled bottom provides a second grinding surface;
placing the solid matrix into the diluent-containing receiving tube;
inserting the plunger into the diluent-containing receiving tube;
grinding the solid matrix between the first grinding surface of the beveled tip and the beveled bottom, thereby releasing molecules embedded in the solid matrix into the diluent for detection.

12. The method of claim 11, wherein the testing device further comprises a test strip positioned in the groove of the plunger, the method further comprising detecting the released molecules in the diluent.

13. The method of claim 12, wherein the test strip is slidably positioned in the groove of the plunger, the method further comprising, after the grinding step, sliding the test strip within the groove until a distal end of the test strip is in contact with the diluent.

14. The method of claim 12, wherein the test strip is a lateral flow immunoassay rapid antigen detection (LFA) test strip.

15. The method of claim 14, wherein the LFA test strip comprises one or more high-affinity binding partners operable to bind and detect an antigen.

16. The method of claim 15, wherein the one or more high-affinity binding partners comprise an antibody.

17. The method of claim 15, wherein the one or more high-affinity binding partners comprise an aptamer.

18. The method of claim 15, wherein the detecting step comprises detecting, with the LFA test strip, one or more diseases or disease-causing antigens selected from the group consisting of Borrelia sp., Ehrlichia sp, Anaplasma sp., Rocky Mountain spotted fever, Rickettsia sp., tularemia, Babesia sp., heartland virus, bourbon virus, Colorado tick fever, Powassan virus disease, relapsing fever, Mediterranean spotted fever, R. conorii, tick-borne encephalitis (TBE Virus), neoehrlichiosis, and Lyme-like illness.

19. The method of claim 15, wherein solid matrix comprises a tick.

20. The testing device of claim 15, wherein the solid matrix comprises a food item or plant and the released and detected molecule comprises Cry9C, exogenous transgenes, small molecules, pesticides, microbial toxins, aflatoxins, universal plant stress proteins, heat-shock proteins, antioxidant enzymes, antimicrobial peptides, delta 8- or delta-9 tetrahydrocannabinol, opium, nicotine or quorum-sensing signals.
